(19) United States
(12) Patent Application Publication
(10) Pub. No.: US 2014/0005547 A1
(43) Pub. Date: Jan. 2, 2014

(54) REMOTELY CONTROLLED ULTRASOUND APPARATUS AND ULTRASOUND TREATMENT SYSTEM

(71) Applicant: General Electric Company, Schenectady, NY (US)

(72) Inventor: Arun Balasubramanian, Bangalore (IN)

(21) Appl. No.: 13/929,474

(22) Filed: Jun. 27, 2013

(30) Foreign Application Priority Data
Jun. 28, 2012 (IN) 2568/CHE/2012

(51) Int. Cl. A61B 8/14; A61B 8/00

(52) U.S. Cl. CPC A61B 8/145; A61B 8/54
USPC 600/447

(57) ABSTRACT

An ultrasound apparatus comprising a sensor assembly configured to be positioned on a patient body portion, the sensor assembly comprises an array of sensors configured to be activated to acquire a plurality of images of the patient body portion, and a remote control unit comprising a detector interface configured to communicate wirelessly with the sensor assembly, and an input device operable by a user to interact with the detector interface, the input device is configured to transmit a plurality of input signals to the sensor assembly, wherein at least one input signal of the plurality of input signals activates at least one sensor of the array of sensors to acquire at least one image of the plurality of images.

### TECHNICAL FIELD OF THE INVENTION

Embodiments of the present invention relate to capturing ultrasound image of a patient body, more specifically to an ultrasound apparatus and an ultrasound treatment system that are remotely controlled.

### BACKGROUND OF THE INVENTION

Medical imaging systems are commonly used for performing diagnostic activities on a patient's body. A commonly used ultrasound based medical imaging system captures images of various body parts of a patient. An ultrasound based medical imaging technique is used to visualize muscles, and many internal organs, for capturing their size, structure and any pathological lesions in addition to any real-time tomographic images. A typical ultrasound imaging system includes an imaging probe that captures images when positioned and moved on a body portion. The imaging probe includes multiple transducer sensors that can generate ultrasound signals as short burst signals. These ultrasound signals are transmitted through the body portion and the ultrasound imaging system waits for return signals during a small time window. The return signals received are then processed to generate multiple images. The ultrasound signals that are not received will be ignored.

Generally to conduct an ultrasound diagnostic procedure, the patient and a medical expert need to be in the same location. The medical expert may be but not limited to a doctor, a technician and a sonographer. The medical expert uses the imaging probe to move over an area of interest on the patient's body for capturing images. If the patient and the medical expert are in different location, in order to conduct the diagnosis on the patient then either the patient or the medical expert needs to travel so that they are in the same location. This may occasionally be time consuming and cumbersome for both the parties. Now considering the case of the patient, the patient may not be having a favorable health condition to travel so reaching a location of an ultrasound imaging system of the medical expert may be nearly impossible.

Moreover trained medical experts are required for capturing the ultrasound images with accuracy. So there may be a non-availability of trained medical experts when a need arises for conducting a diagnosis especially in rural regions. In order to address these situations ultrasound images are captured and then compressed and transferred over a network to the trained medical expert in a remote location. As the medical expert do not have control over the ultrasound imaging system, the medical expert examines these images and then provides suggestion and instructions to a local technician for taking other ultrasound images if required. These images taken are then sent to the medical expert for examination which renders the process time consuming and cumbersome.

Thus there is a need for an ultrasound apparatus that conveniently captures the images of the patient for examination by a medical expert in a remote location.

### BRIEF DESCRIPTION OF THE INVENTION

The above-mentioned shortcomings, disadvantages and problems are addressed herein which will be understood by reading and understanding the following specification.

As discussed in detail below, embodiments of the present invention include an ultrasound apparatus for capturing a medical image of a patient. The ultrasound apparatus is remotely controlled by a medical expert The ultrasound apparatus includes a sensor assembly and a remote control unit. The sensor assembly is configured to be positioned on a patient body portion. The sensor assembly comprises an array of sensors configured to activate for acquiring multiple images of the patient body portion. The remote control unit comprises a detector interface capable of communicating wirelessly with the sensor assembly. A user operates an input device for interacting with the detector interface. The user may be a medical expert such as, a doctor or a sonographer or a technician. During interaction the input device transmits multiple input signals to the sensor assembly. One or more input signals activate one or more sensors for capturing one or more images of the patient body portion.

In an embodiment, an ultrasound treatment system for capturing a medical image of a patient is disclosed. The ultrasound treatment system includes a remote control unit and a sensor control unit. The remote control unit includes a detector module for receiving multiple input signals from the user through a detector interface. The input received activates one or more sensors of an array of sensors positioned proximal to the patient body. These multiple input signals are transmitted to the sensor control unit using the transceiver. The transceiver unit of the sensor control unit receives the multiple input signals over a wireless network. The sensor module activates one or more sensors of the array of sensors based on the received input signals. The signals transmitted by the sensors are used for generation of multiple images of the patient body portion.

In an embodiment, a method for remotely controlling an ultrasound procedure on a patient body is disclosed. The method includes receiving multiple input signals over a wireless network in response to interaction of an input device on a detector interface. The input device is operated by a user on the detector interface present in a remote location. Thereafter, one or more sensors of an array of sensors positioned proximal to the patient body are activated based on the multiple input signals. These activated sensors capture one or more images of the patient body portion.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of an ultrasound apparatus for capturing a medical image of a patient body portion in accordance with an embodiment;

FIG. 2 is a schematic illustration of exemplary environment wherein an ultrasound apparatus operates for capturing images of the patient's body portion in accordance with an embodiment;

FIG. 3 is a schematic illustration of a detector interface of an ultrasound apparatus in accordance with an embodiment;

FIG. 4 is a schematic illustration of a sensor assembly of an ultrasound apparatus in accordance with an embodiment;

FIG. 5 is a schematic illustration of an exemplary environment wherein a display unit presents a model in accordance with an embodiment;

FIG. 6 is a schematic illustration of a ultrasound treatment system in accordance with an embodiment;

FIG. 7 illustrates a flowchart of a method of remotely controlling an ultrasound procedure on a patient body in accordance with an embodiment; and

FIG. 8 illustrates a flowchart of a method of remotely controlling an ultrasound procedure on a patient body in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In an embodiment an ultrasound apparatus for capturing a medical image of a patient is disclosed. The ultrasound apparatus is remotely controlled by a medical expert. The ultrasound apparatus includes a sensor assembly and a remote control unit. The sensor assembly is configured to be positioned on a patient body portion. The sensor assembly comprises an array of sensors configured to activate for acquiring multiple images of the patient body portion. The remote control unit comprises a detector interface capable of communicating wirelessly with the sensor assembly. A user operates an input device for interacting with the detector interface. The user may be a medical expert such as, a doctor or a sonographer or a technician. During interaction the input device transmits multiple input signals to the sensor assembly. One or more input signals activate one or more sensors for capturing one or more images of the patient body portion.

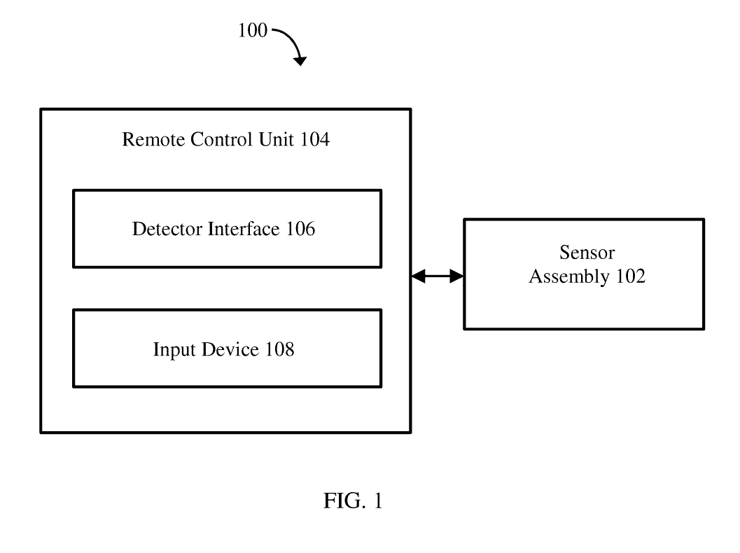

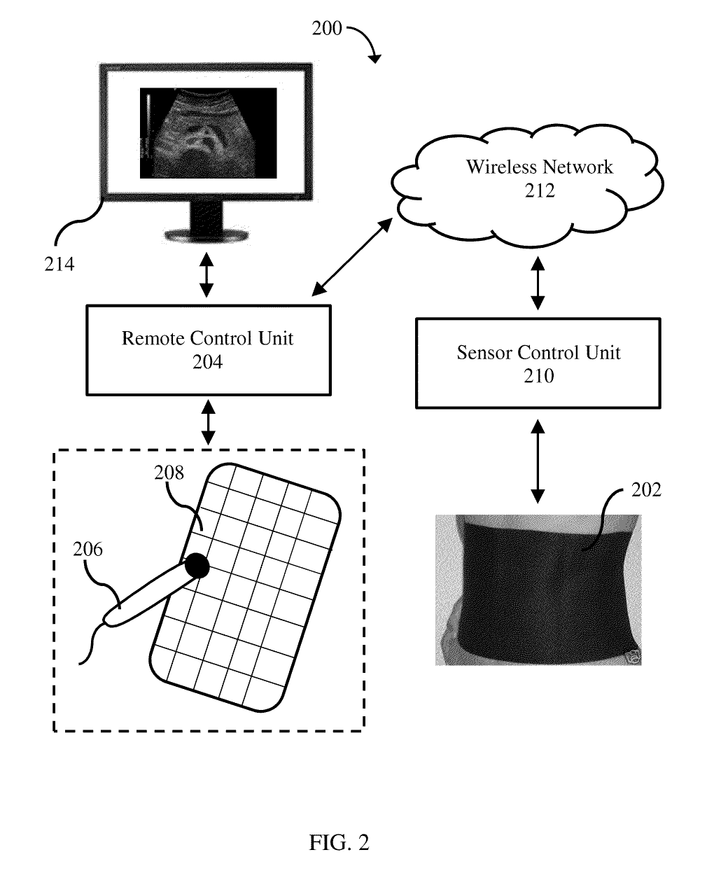

FIG. 1 is a schematic illustration of an ultrasound apparatus 100 for capturing an image of a patient body portion in accordance with an embodiment. The ultrasound apparatus 100 may be used for sending ultrasound signals to a patient body to capture an image of a region of interest. The region of interest may be a portion of the body or a body portion that needs to be focused for capturing an image. The region of interest may be determined by a doctor based on a body portion that needs to be diagnosed. The patient body portion may include for example an abdomen and a chest. The image may be examined by the doctor or any other medical practitioner to determine any variation in health condition of the patient. The ultrasound apparatus 100 may be operated by a user such as the medical expert. The medical expert may include but not limited to a doctor, a sonographer or a technician. The medical expert may be present in a remote location operating the ultrasound apparatus 100. This is explained in detail in conjunction with FIG. 2.

The ultrasound apparatus 100 comprises a sensor assembly 102 configured to be positioned on the patient body portion. The sensor assembly 102 may include an array of sensors. The sensors may be for example ultrasound transducers. In an embodiment, the sensor assembly 102 may be wearable by the patient. The sensor assembly 102 may be worn to cover the patient body portion that needs to be diagnosed. The sensor assembly 102 may be in the form of a belt like structure having a fastening unit to be wearable on the patient's body. In an embodiment, the sensor assembly 102 may include multiple separate units of sensors that may be placed on different positions on the patient's body. Each unit of sensor may include one or more sensors. Each unit of sensor may have any fastening mechanism for adhering to the patient's body. The array of sensors in the sensor assembly 102 may be activated to acquire one or more images of the body portion. The activation or operation of the sensors is controlled by the user through a remote control unit 104. The remote control unit 104 includes a detector interface 106 and an input device 108 capable of interacting with the detector interface 106. The detector interface 106 may be but is not limited to a hardware detector interface and a virtual detector interface. The input device 108 may be operated by the user. The user moves the input device 108 with respect to the detector interface 106 for initiating an interaction. This interaction results in generation of multiple input signals that may be transmitted to the sensor assembly 102. The input signals facilitate in activating one or more sensors of the array of sensors. A position of interaction of the input device 108 on the detector interface 106 may determine a sensor that needs to be activated. For example if input device 108 interacts to cover an area in the detector interface 106, then sensors corresponding to the covered area from the array of sensors may be activated. The activated sensors send ultrasonic signals to the patient's body portion and subsequently reflected signals may be processed to generate one or more images. The images are then presented to the medical expert.

Now referring to FIG. 2 schematically illustrating an exemplary environment 200 wherein an ultrasound apparatus such as the ultrasound apparatus 100 operates for capturing images of the patient's body portion in accordance with an embodiment. As shown in FIG. 2, a sensor assembly 202 may be worn by the patient. The sensor assembly 202 illustrated may be a belt like structure that may be fastened to the patient's body portion such as abdomen. In an embodiment, the sensor assembly 202 may include a fastening unit that may be used for positioning the sensor assembly 202 on the patient's body portion. The fastening unit may include for example a Velcro®, a clip unit and a hook unit. However, it is contemplated that other embodiments may include various other fastening units for positioning the sensor assembly 202 on the patient body portion. The sensor assembly 202 includes an array of sensors that may be capable of generating ultrasonic signals for capturing the images of the patient's body portion.

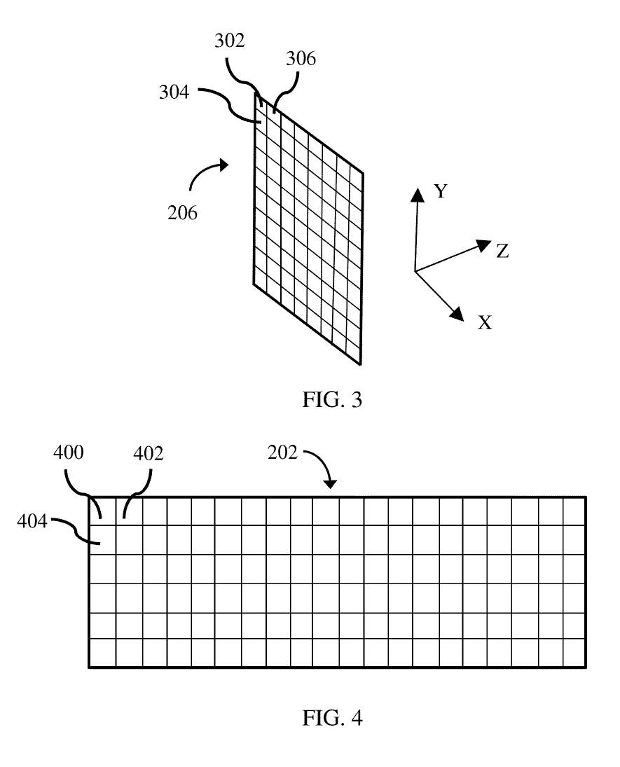

In order to send out the ultrasonic signals the sensors need to be activated. The sensors may be activated based on a user input. The user input may be received through a remote control unit 204. The remote control unit 204 receives instructions for activating the sensors in response to an interaction between an input device 206 and a detector interface 208. The remote control unit 204, the input device 206 and the detector interface 208 may be present in a remote location. The user may move the input device 206 with respect to the detector interface 208. In an embodiment the detector interface 208 may have a grid configuration. As illustrated in FIG. 2 the grid configuration includes multiple cells, Each cell may represent or correspond to a set of sensors of the array of sensors in the sensor assembly 202. For example if the user selects a cell on the detector interface 208 then a corresponding set of sensors from the sensor assembly 202 may be activated. This is explained in detail in conjunction with the embodiments presented in FIG. 3 and FIG. 4. In order to activate the set of sensors, multiple input signals may be generated by the remote control unit 204. The remote control unit 204 may be an integral part of one of the detector interface 208 and the input device 206. Alternatively, the remote control unit 204 may be an external unit connected to one of the detector interface 208 and the input device 206. The remote control unit 204 transmits the multiple input signals to a sensor control unit 210. The multiple input signals are transmitted over a wireless network 212. The wireless network 212 may include but are not limited to, a 3^{rd} Generation communication (3G) network, a 4 Generation communication (4G) network, and a Long Term Evolution communication (4G LTE) network. The wireless network 212 facilitates transmission of the input signals with minimum time lag so that these signals are transmitted in real-time. The sensor control unit 210 receives and processes these input signals to activate the corresponding sensors in the sensor assembly 202. The activated sensors send ultrasonic signals to the patient's body portion and receives back reflected signals. Then the reflected signals may be transmitted to an image processing unit present in the remote location over the wireless network 212. The image processing unit generates the images in real-time and presents to the user in a display unit 214. The images are presented in a continuous stream with minimum time lag thereby providing a real-time viewing experience to the user. In an embodiment, the image processing unit may be present in the location of the patient and generates the images from the reflected signals. These images may be transmitted over the wireless network 212 for presenting through the display unit 214.

Now considering an embodiment wherein the detector interface 208 may be a hardware detector interface, the input device 206 may be moved maintaining contact with the detector interface 208. The input device 206 may be moved to contact one or more cells in the detector interface 208. While maintaining contact input signals are generated for activating sensors in the sensor assembly 202. Further the input device 206 may be used to apply pressure on the detector interface 208. This pressure may be translated into generation of higher intensity signals from the sensors. For example a detector interface may be a pad like structure having a grid configuration. The pad like structure may be but is not limited to, a two dimensional structure and a three dimensional structure. A sonographer or a technician may move an input device on the detector interface to select one or more cells of interest. Once the cells are selected then input signals are sent by the detector interface to a sensor control unit. Alternatively the input device sends the input signals to the sensor control unit. Consequently corresponding sensors present in the sensor assembly are activated based on the input signals. The activated sensors send ultrasonic signals.

The ultrasonic signals transmitted by the sensors may be of different frequencies or intensities. The intensity of the ultrasonic signals depends on a pressure applied by the input device on the detector interface. More specifically, when the pressure applied on a cell of the one or more interested cells by the input device is greater, then the intensity of ultrasonic signals generated by the corresponding sensors of the cell may be higher. When the intensity of the ultrasonic signals is higher the images generated may have higher resolution. Moreover, in an embodiment, a direction of application of pressure on the detector interface may determine a direction of transmission of the ultrasonic signals by the activated sensors to the patient's body portion. This is explained in detail in conjunction with the embodiments depicted in FIG. 3 and FIG. 4.

In an embodiment, the detector interface 208 may be a virtual detector interface. The virtual detector interface may be presented to the user. The user may employ the input device 206 to perform gestures for selecting one or more cells of a grid configuration in the detector interface 208. The gestures may include but are not limited to moving or maneuvering the input device 206 in space in multiple axes. A direction of movement in the multiple axes determines a direction in which the ultrasonic signals need to be transmitted from the interested sensors to the patient body portion. Further a velocity or a speed of movement of the input device 206 with respect to the detector interface 208 may be translated into an intensity of ultrasonic signals transmitted by the interested sensors. The detector interface 208 may be configured to detect these movements of the input device 206 and then send input signals to the sensor control unit 210 for activating the corresponding sensors.

Turning now to an embodiment depicted in FIG. 3, FIG, 3 illustrates a detector interface 208 of an ultrasound apparatus in accordance with an embodiment. The detector interface 208 includes multiple cells for example, a cell 302, a cell 304 and a cell 306. Each cell corresponds to one or more sensors present in the sensor assembly 202 as illustrated in FIG. 4. For example, a cell 302 corresponds to a sensor 400, a sensor 402 and a sensor 404. Similarly each other cell may correspond to multiple sensors thereby all the cells in the detector interface 208 cover all the sensors in the sensor assembly 202. As the sensor assembly 202 is worn by the patient, selection of cells results in activation of sensors present proximal to different body portion. The sensor assembly 202 may be a two dimensional assembly or a three dimensional assembly.

During operation the user may position the input device 206 on the detector interface 208 and selects for example, the cell 302. Consequently a position of the cell 302 may be identified with respect to multiple axes. For instance the position of the cell 302 may be determined with respect to X and Y axes. This position information of the cell 302 may be send to the sensor control unit 102 and subsequently the sensor control unit 102 process the position information to determine that the cell 302 is selected. As a result, corresponding with sensor 400, the sensor 402 and the sensor 404 may be activated. The sensor control unit 102 may include information indicating mapping between the sensor 400, the sensor 402 and the sensor 404 and the cell 302. Similarly mapping information between other cells and the corresponding sensors may be stored in the sensor control unit 102. The sensor control unit 102 may have a memory for storing this mapping information. In an alternate embodiment one of the detector interface 208 and the input device 206 may store the mapping information and send the information to the sensor control unit 102 based on the cell selected. In this case the detector interface 208 and the input device 206 may have a memory.

Further, in an embodiment an orientation of the input device 206 with respect to the detector interface 208 may determine the direction of transmission of the ultrasonic signals from the sensor 400, the sensor 402 and the sensor 404. For instance, the input device 206 may be oriented at an angle respect to the Y axis and the angular orientation information may be transmitted to the sensor control unit 102. The sensor control unit 102 processes this information and activates the corresponding sensors for example, the sensor 400 to transmit the ultrasonic signal into the patient's body portion in the oriented angle. When ultrasonic signals are transmitted from different angles, the ultrasonic signals reflected from tissues of the patient's body portion may be received and processed to generate images of the tissues from different angles. This also facilitates in improved diagnosis of the tissues.

Moreover the ultrasonic signals may be transmitted to different depths of the body portion to capture more detail images of various tissues. Hence the input device 206 may be pressed against the detector interface 208 so that the pressure applied may be sensed by the detector interface 208. Then the remote control unit 204 processes the information associated with the pressure applied to activate the corresponding sensors such as sensor 400, a sensor 402 and a sensor 404 in the sensor assembly 202. In this embodiment, a pressure applied on the detector interface 208 may be measured and corresponding intensity level at which the ultrasonic signals need to be generated may be identified. Then information regarding the intensity level may be communicated to the sensor control unit 210 for activating corresponding sensors to generate ultrasonic signals at the intensity level. The ultrasonic signals transmitted at higher intensity reach higher depths of the patient's body portion. The ultrasonic signals reflected back from various tissues of the patient's body portion may be processed by the sensor control unit 210. The processed signals may be converted into images by an image processing unit (not shown in the figures) and presented to the medical expert through the display unit 214.

In an embodiment, the detector interface 208 may be a three dimensional interface. For example a detector interface 208 may have a shape of a body portion such as but not limited to a limb and a body part. However, it is contemplated that other embodiments of the detector interface 208 may have any other shapes. In this scenario, the input device 106 may be positioned on a portion of the detector interface 208 and sensors proximal to corresponding portion of the patient's body may be activated. For example, if an input device is positioned on a palm portion of a detector interface having the shape of the hand then sensors proximal to a palm portion of the patient's hand are activated. The sensors may transmit ultrasonic signals to capture the image of the palm portion. Multiple ultrasonic signals may be transmitted for time duration till the input device is in contact with the detector interface. Reflected signals received from the palm portion may be processed to generate real time images of the palm portion.

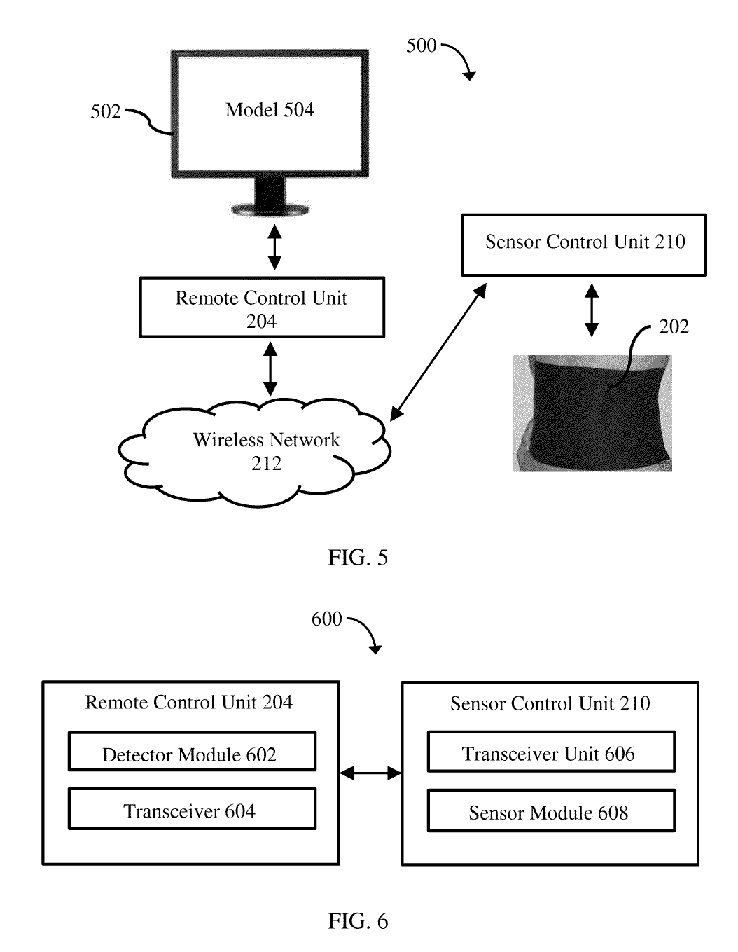

In an embodiment, a display unit may act as a detector interface and receive user input. FIG. 5 is a schematic illustration of an exemplary environment 500 wherein a display unit 502 presents a model 504 in accordance with an embodiment. The model 504 may include a grid configuration including multiple cells. The cells may be selected by the user using the input device. In an embodiment the display unit 502 may include a touch sensitive interface and accordingly the user may use the input device or hand to select interested cells in the model.

The model may include but is not limited to a two dimensional model and a three dimensional model. The model may be a two dimensional structure similar to the pad like structure shown in FIG. 2. In this case the user may select few interested cells and consequently corresponding sensors in the sensor assembly 202 may be activated. In an embodiment the model may be a three dimensional model of a patient's body portion such as, a chest, an abdomen and a hand. The user may select interested cells from the three dimensional model. For example, the user may select interested cells on a three dimensional model of an abdomen. The selected cells correspond to a region of interest on a patient's abdomen. The region of interest may be decided by a doctor or a medical expert depending on a body portion of the patient that needs to be diagnosed. Then the input signals are transmitted by the remote control unit 204 to the sensor control unit 210 connected to the sensor assembly 202 over the wireless network 212. The sensor control unit 210 activates sensors corresponding to the interested cells for transmitting ultrasonic signals to the region of interest of the patient's body portion.

Turning now to FIG. 6, an ultrasound treatment system 600 in accordance with an embodiment is schematically illustrated. The ultrasound treatment system 600 includes the remote control unit 204 and the sensor control unit 210 communicating over the wireless network 212. The remote control unit 204 includes a detector module 602 for receiving multiple inputs from the user. The inputs are received in response to an interaction between the detector interface 208 and the input device 106 operated by the user. The interaction includes selection of one or more cells of the multiple cells present in the detector interface 208. The inputs include the position information of the selected cells in the multiple axes such as X and Y axes. In an embodiment the detector module 602 determines the position information of the selected cells communicated by the input device 106. Thereafter the detector module 602 transmits multiple input signals through a transceiver 604. The input signals may include information associated with one or more sensors to be selected. In this scenario the detector module 602 maps the position of interaction of the input device 106 on the detector interface 208 with the one or more sensors of the array of sensors present in the sensor assembly 202. This is performed by determining one or more coordinate positions of the input device on one or more cells of the detector interface 208. Then the coordinate positions are associated with positions of the one or more sensors corresponding to the selected cells. In an embodiment, the input signals may include the position information of the selected cells.

The input signals are received by a transceiver unit 606 in the sensor control unit 210 over the wireless network 212. The sensor module 608 processes the input signals to activate the one or more sensors of the array of sensors present in the sensor assembly 202. In case the input signals include information related to the sensors then the sensor module 608 directly activates the sensors. In an alternate embodiment, if the input signals include the position information of the selected cells, then the sensor module 608 determines position information of the corresponding sensors. A mapping between the position of the cells and the position of the sensors may be stored in the sensor control unit 210. Subsequently the sensor module 608 activates the sensors. The sensors then send the ultrasonic signals to the patient body portion. The signals may be transmitted in different directions based on the orientation and interaction of the input device 106 with respect to the detector interface 208. Further, the ultrasonic signals send may have one or more focus attributes. The focus attributes may include but are not limited to the frequency of the ultrasonic signals and the depth at which the ultrasonic signals are transmitted to the patient's body portion. The focus attributes may vary dependent on the interaction of the input device 106 with the detector interface 208 in the multiple axes such as, a Z axis. Once the signals are received at the patient body portion they are reflected and received at the sensor control unit 210. The sensor control unit 210 may process the reflected signals and send to the image processing unit. Thereafter the image processing unit processes the reflected signals to generate the images.

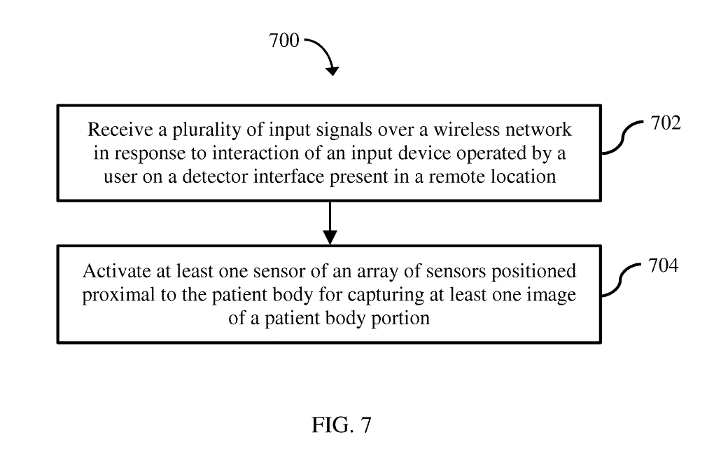

Now turning to an embodiment depicted in FIG. 7 showing a flowchart of a method 700 of remotely controlling an ultrasound procedure on a patient body. In this method at step 702 multiple input signals are received over a wireless network in response to interaction of an input device on a detector interface. The input device is operated by the user on the detector interface present in the remote location. The detector interface may have a grid configuration and accordingly multiple cells may be present in the detector interface. So during interaction the input device may be used to select one or more cells. The one or more cells correspond to one or more sensors of the array of sensors in a sensor assembly. The sensor assembly may be worn by the patient present in another location. In another embodiment the input signals may include the position information of the selected cells.

Once the interested cells are selected then one or more sensors of the array of sensors may be activated at step 704. The activated sensors proximal to a region of interest of the patient body portion send ultrasonic signals. The signals reflected from the patient body portion may be processed to obtain one or more images of the patient body portion.

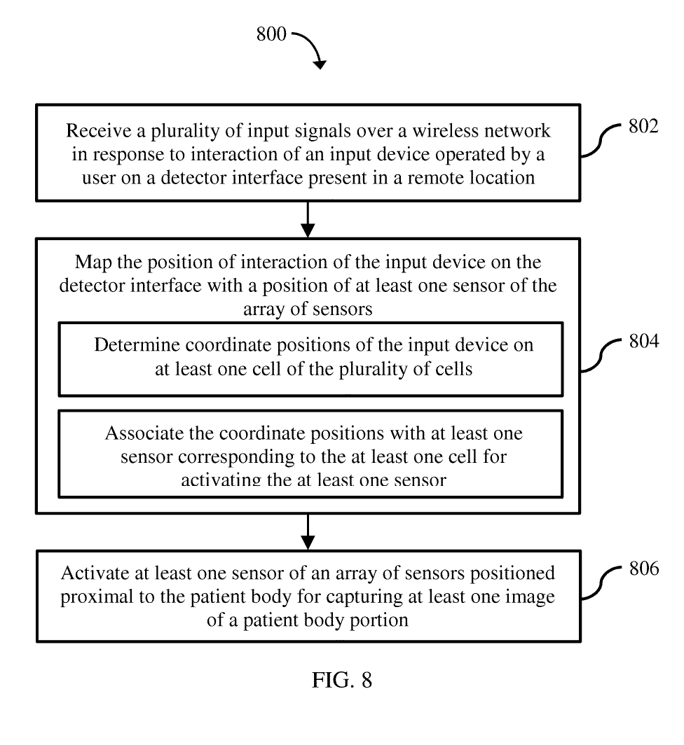

FIG. 8 is a flowchart of an embodiment of a method 800 for remotely controlling an ultrasound procedure on a patient body. In this method at step 802 multiple input signals are received over a wireless network in response to interaction of an input device on a detector interface. The input device is operated by the user on the detector interface present in the remote location. The detector interface may have a grid configuration and accordingly multiple cells may be present in the detector interface. So during interaction the input device may be used to select one or more cells. The one or more cells correspond to one or more sensors of the array of sensors in a sensor assembly. The sensor assembly may be worn by the patient present in another location. In an embodiment the position of interaction of the input device on the detector interface is mapped with a position of the one or more sensors of the array of sensors at step 804. This is performed by determining one or more coordinate positions of the input device on the one or more cells of the detector interface. Then the coordinate positions are associated with positions of the one or more sensors corresponding to the selected cells. In an embodiment, the input signals may include the position information of the selected cells.

Once the interested cells are selected then one or more sensors of the array of sensors may be activated at step 806. The activated sensors proximal to a region of interest of the patient body portion send ultrasonic signals. The signals reflected from the patient body portion may be processed to obtain one or more images of the patient body portion.

The methods 700 and 800 can be performed using a processor or any other processing device. The method steps can be implemented using coded instructions (e.g., computer readable instructions) stored on a tangible computer readable medium. The tangible computer readable medium may be for example a flash memory, a read-only memory (ROM), a random access memory (RAM), any other computer readable storage medium and any storage media. Although the method of remotely controlling an ultrasound procedure on a patient body is explained with reference to the flow chart of FIGS. 7 and 8, other methods of implementing the method can be employed and are contemplated. For example, the order of execution of each method steps may be changed, and/or some of the method steps described may be changed, eliminated, divide or combined. Further the method steps may be sequentially or simultaneously executed for remotely controlling an ultrasound procedure on a patient body.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An ultrasound apparatus comprising:
a sensor assembly configured to he positioned on a patient body portion, the sensor assembly comprises an array of sensors configured to be activated to acquire a plurality of images of the patient body portion; and
a remote control unit comprising:
a detector interface configured to communicate wirelessly with the sensor assembly; and
an input device operable by a user to interact with the detector interface, the input device is configured to transmit a plurality of input signals to the sensor assembly, wherein at least one input signal of the plurality of input signals activates at least one sensor of the array of sensors to acquire at least one image of the plurality of images.

2. The ultrasound apparatus of claim 1, wherein the sensor assembly is wearable by a patient.

3. The ultrasound apparatus of claim 1, wherein the detector interface is one of a hardware detector interface and a virtual detector interface.

4. The ultrasound apparatus of claim 1, wherein the detector interface presents a multi-dimensional model of the patient body portion, wherein an input signal of the plurality of input signals comprises information associated with a position of interaction of the input device on the multidimensional model, wherein the position of interaction corresponds to a position on the patient body portion.

5. The ultrasound apparatus of claim 1, wherein the detector interface comprises a grid configuration comprising a plurality of cells, wherein a cell of the plurality of cells represents a set of sensors of the array of sensors.

6. The ultrasound apparatus of claim 5, wherein the grid configuration is dynamically reconfigurable, and the detector interface is a virtual detector interface.

7. The ultrasound apparatus of claim 5, wherein an input signal of the plurality of input signals comprises information associated with a position of interaction of the input device on the detector interface, the position of interaction is indicated by coordinate positions in a plurality of axes.

8. The ultrasound apparatus of claim 7, wherein the remote control unit is configured to map the position of interaction of the input device on the detector interface with a position of the at least one sensor, wherein the remote control unit is configured to map the position of interaction by:
determining at least one coordinate position of the input device on at least one cell of the detector interface; and
associating the at least one coordinate position of the input device with positions of at least one sensor corresponding to the at least one cell to activate the at least one sensor corresponding to the at least one cell.

9. The ultrasound apparatus of claim 7, wherein a movement of the input device in the plurality of axes during interaction with the detector interface corresponds to at least one of:
at least one direction of transmission of ultrasonic signals from a sensor of the at least one sensors to the patient body portion to acquire the at least one image of the patient body portion; and
at least one focus attribute associated with the transmitted ultrasonic signals with respect to the patient body portion.

10. The ultrasound apparatus of claim 1 further comprising a display device communicably coupled to the remote control unit and configured to present the plurality of images to the user.

11. An ultrasound treatment system comprising:
a remote control unit comprising:
a detector module configured to receive a plurality of inputs from a user through a detector interface, wherein an input of the plurality of inputs is for activating at least one sensor of an array of sensors positioned proximal to a patient body portion; and
a transceiver configured to transmit a plurality of input signals corresponding to the plurality of inputs; and
a sensor control unit comprising:
a transceiver unit configured to receive the plurality of input signals over a wireless network; and
a sensor module configured to activate the at least one sensor of the array of sensors based on the received plurality of input signals, wherein the array of sensors is configured to acquire a plurality of images of the patient body portion.

12. The ultrasound treatment system of claim 11, wherein the detector module is further configured to map a position of interaction of an input device on the detector interface with a position of at least one sensor of the array of sensors, wherein the input device is operable by the user.

13. The ultrasound treatment system of claim 12, wherein an input signal of the plurality of input signals comprises information associated with the position of interaction, wherein the position of interaction is indicated by coordinate positions in a plurality of axes.

14. The ultrasound treatment system of claim 13, wherein the detector interface comprises a grid configuration comprising a plurality of cells, a cell of the plurality of cells represents a set of sensors of the array of sensors, wherein the detector module is configured to map the position of interaction by:
determining coordinate positions of the input device on at least one cell of the detector interface; and
associating the coordinate positions of the input device with at least one sensor corresponding to the at least one cell for activating the at least one sensor corresponding to the at least one cell.

15. The ultrasound treatment system of claim 13, wherein based on a movement of the input device in the plurality of axes during interaction with the detector interface, the sensor module is further configured to define at least one of:
at least one direction of transmission of ultrasonic signals from a sensor of the at least one sensors to the patient body to acquire at least one image of the patient body portion; and
at least one focus attribute associated with the transmitted ultrasonic signals with respect to the patient body portion.

16. A method of remotely controlling an ultrasound procedure on a patient body, the method comprising:
receiving a plurality of input signals over a wireless network in response to interaction of an input device operated by a user on a detector interface present in a remote location; and
activating at least one sensor of an array of sensors positioned proximal to the patient body to acquire at least one image of a patient body portion, wherein the at least one sensor is activated based on at least one received input signal of the plurality of input signals.

17. The method of claim 16, wherein an input signal of the plurality of input signals comprises information associated with a position of interaction of the input device on the detector interface, the position of interaction is indicated by coordinate positions in a plurality of axes.

18. The method of claim 17, further comprising mapping the position of interaction of the input device on the detector interface with a position of the at least one sensor of the array of sensors.

19. The method of claim 18, wherein the detector interface comprises a grid configuration comprising a plurality of cells, wherein a cell of the plurality of cells represents a set of sensors of the array of sensors, and wherein mapping the position of interaction of the input device comprises:
determining coordinate positions of the input device on at least one cell of the plurality of cells; and
associating the coordinate positions of the input device with at least one sensor corresponding to the at least one cell for activating the at least one sensor corresponding to the at least one cell.

20. The method of claim 17, further comprising defining, based on a movement of the input device in the plurality of axes during interaction with the detector interface, at least one of:
at least one direction of transmission of ultrasonic signals from a sensor of the at least one ultrasonic sensors to the patient body portion to acquire at least one image of the patient body portion; and
at least one focus attribute associated with the transmitted ultrasonic signals with respect to the patient body portion.
